(19) United States
(12) Patent Application Publication
(10) Pub. No.: US 2019/0117417 A1
(43) Pub. Date: Apr. 25, 2019

(54) BIDIRECTIONAL LIMB NEURO-PROSTHESIS

(71) Applicant: ECOLE POLYTECHNIQUE FEDERALE DE LAUSANNE (EPFL), Lausanne (CH)

(72) Inventors: Stanisa RASPOPOVIC, Lausanne (CH); Francesco Maria PETRINI, Lausanne (CH); Marco CAPOGROSSO, Pully (CH); Marco BONIZZATO, Lausanne (CH); Silvestro MICERA, Saint-Sulpice (CH)

(21) Appl. No.: 16/165,883

(22) Filed: Oct. 19, 2018

Related U.S. Application Data
(62) Division of application No. 15/107,108, filed on Jun. 22, 2016.

(30) Foreign Application Priority Data
Dec. 23, 2013 (IB) PCT/IB2013/061286
Sep. 4, 2014 (CH) 01340/14

(51) Int. Cl. A61F 2/72; A61F 2/60; A61B 5/04; A61F 2/54; A61F 2/78; A61N 1/05

(52) U.S. Cl. CPC A61F 2/72; A61F 2/60; A61B 5/04001; A61B 5/04004; A61F 2002/6872; A61F 2/7812; A61N 1/0551; A61F 2002/543; A61F 2002/607; A61F 2/54

(57) ABSTRACT

Integrated closed-loop real-time limb neuro-prosthetic system comprising an artificial limb, a microprocessor, sensors, a signal conditioner, a stimulator, at least one EMG electrode and at least one sensory feedback electrode, characterized by the fact that said sensory feedback electrode is all intraneural electrode which is adapted to be implanted in an intact and healthy portion of a nerve.

### FIELD OF INVENTION

The invention relates to limb neuro-prostheses.

### BACKGROUND

Dexterous manipulation by upper limb and skillful walking, running or jumping in lower limb are achieved through a complex relationship between motor commands, executed movements, and sensory feedback during limb activities. Limb loss causes severe physical debilitation and often distress. An ideal prosthesis should reproduce the bidirectional link between the user's nervous system and the peri-personal environment by exploiting the post-amputation persistence of the central and peripheral neural networks and pathways devoted to hand motor control [1] and sensing [2-5].

In the case of upper limb, skillful object grasping and manipulation is compromised, thus depriving the person of the most immediate and important source of tactile sensing in the body. For these reasons, replacing a lost hand and its precise functionalities is a major unmet clinical need that is receiving attention from engineers, neurophysiologists, and clinicians among the others.

In particular, real-time and natural feedback from the hand prosthesis to the user is essential to enhance the control and the functional impact of prosthetic hands in daily activities, prompting their full acceptance by users within an appropriate “body scheme” that does not require continuous visual monitoring, as with current artificial hands [6,7]. Recent notable advances in the field of hand prostheses have included designing devices with multiple degrees of freedom and equipped with different sensors [8-10]. These developments have made the need for effective bidirectional control even more compelling. A promising solution is represented by targeted muscle reinnervation [TMR], which consists of rerouting the residual nerves of the amputees over the chest muscles [11, 12]. Individuals with arm or hand amputations can chronically use TMR-based prostheses, which could theoretically allow for a certain amount of sensory feedback [13, 14]. However, because the superficial electromyogram (sEMG), used as a control signal, is recorded from the same body region (i.e., the chest) that must be mechanically stimulated to provide feedback, real-time bidirectional control could be difficult to achieve. In this scenario, TMR subjects must contract muscles and simultaneously perceive a touch sensation on the skin overlying the same muscles, therefore possibly producing the so-called neurophysiological “sensory gating” [15].

In the case of lower limb amputation, especially in the higher level ones, the control is very limited, and often requires big effort from user, while the feedback is completely absent. With absence of the sensory feedback tasks like maintaining balance or walking symmetrically become much more challenging, while stepping over unexpected surfaces or obstacles become close to impossible. Analogously as in the upper limb, recently, the promising solution is proposed by TMR, where EMG signals were decoded and combined with data from sensors on the prosthesis to interpret the patient's intended movements [16].

Sensory feedback can be restored to amputees by means of non-invasive techniques. Mechanical (i.e. vibration) stimulation of the skin over the forearm or the arm has been driven by tactile and angular information from a robotic hand [28]. This approach, however, requires a training (eventually long) for the amputees in order to learn the sensory feedback code (how the prosthesis information are transduced into the mechanical stimulation modulation), which is not homologous (there is the necessity for the interpretation of the given stimulation).

Another way to restore sensory feedback to amputees is the electrical stimulation of the human extremities peripheral nerves by means of electrodes placed on the skin (Transcutaneous Electrical Nerve Stimulation (TENS)). Indeed, by this kind of stimulation, tactile sensations can be elicited over the phantom hand (or foot) of an amputee [29]. TENS causes an activation of most of the sensory fibers simultaneously (low selectivity) [30] but does not require training for the patient because the stimulation is homologous.

As a separate matter, the rapid development of neural interfaces for the peripheral nervous system [17] has provided potential for new tools through which bidirectional communication with residual nerves post-amputation could be potentially restored. Initial feasibility demonstrations of the induction of some sensations [18] and preliminary trials of the sporadic control of non-attached prostheses [19-21] have recently been performed in the upper limb.

U.S. Pat. No. 7,302,296B1 discloses the possibility of sensory restoration in amputees using epineural (disposed outside and around the nerve) cuff electrodes with frequency modulation. Cuff electrodes are known to be prone to a poor selectivity that can cause the impossibility of the modulation of a localized sensation [17, 22].

US patent application US 2013/253606 discloses a peripheral nerve interface system which may control a prosthetic hand. This system requires the use of an element named nerve conduit to establish a connection between the prosthetic hand and the damaged peripheral nerve. In order that the connection between the interface (the nerve conduit) and the nerve is successfully built, the nerve itself has to re-grow after damage, which should be eventually induced. This is a very aggressive approach, hence, the biocompatibility is critical in order to guarantee the longevity of such an interface (e.g. the nerve can be irreversibly damaged by cut).

### GENERAL DESCRIPTION OF THE INVENTION

The goal of the invention is to address the problems mentioned in the previous chapter related to the bidirectional control and especially sensory feedback in limb prostheses.

Those problems are solved with the system defined in the claims. According to a preferred embodiment of the invention, they are solved by the use of multi- and intra-fascicular intraneural (within the nerve) electrodes [17] that can achieve superior performance [22] with combined charge, frequency and temporal modulations. In fact, thanks to the high selectivity (capacity to stimulate desired fibers without eliciting non targeted fibers) of multi- and intra-fascicular intraneural electrodes it is possible to design device that implements innovative sensory feedback modulation strategies.

Multi- and intra-fascicular intraneural electrodes can achieve a sufficient precision in fiber recruitment being able to selectively activate motor (and sensory) fiber groups even in the same fascicle [23] by modulating injected charge. Therefore, it could be possible to elicit realistic sensations by recruiting a proper fibers population (encoding) by modulating the charge, and/or the frequency and/or the time occurrence of the stimulation.

These electrodes are implanted transversally in the nerve in order to take into account its anatomical and functional organization. In particular, the neural fibers within the nerve are organized in fascicles that bring specific information from the extremities of the body to the brain and vice versa. In FIG. 1 are reported an example of implantation of a multi- and intra-fascicular intraneural electrode in a nerve and a drawing representing the anatomy and the functional organization of the median nerve.

In order to complete and fully integrate the restored sensory pathway in the user control strategy the nerves stimulation may be advantageously combined in real-time (unperceivable delay [24]) to a hybrid Electromyographic (EMG)/Electroneurographic (ENG) control system to achieve the novel concept of a full bidirectional limb prosthesis, that would interact and adapt to each specific user natural control strategy.

The present invention therefore consists of an integrated real-time limb neuro-prosthetic system comprising a microprocessor with implemented range of strategies for nerve stimulation and movement intention decoding, an artificial limb, sensors, EMG and sensory feedback electrodes, a signal conditioner and a stimulator.

The system according to the invention preferably includes at least one multi- and/or one intrafascicular intraneural electrode.

The following additional features are comprised in different embodiments of the invention:
Injected charge, stimulation frequency, and time occurrence of the stimulation patterns real-time modulation in order to elicit a natural sensation with multi- and intra-fascicular intraneural electrodes.
Multipolar stimulation to control the position and/or type of sensation and its extension over the phantom limb (e.g. touch/pressure/proprioception).
Real time hybrid decoding (from EMG and/or ENG signals) of grasps/movements and their velocity and force.
Real time integration of motor commands and sensory restoration modules in a prosthetic limb controller.

The basis of the invention, in particular the condition for the above-cited four points to work synergistically is that multi- and intra-fascicular intraneural electrodes are provided as an interface with the peripheral nervous system for the design of a bidirectional neuroprosthesis aiming at substituting a missing limb. An example of this kind of electrodes is represented by TIMEs [25].

More generally the present invention encompasses a system and methods implemented to create a bidirectional neuroprosthesis able to restore the lost limb motor and sensory functions in upper or lower limb amputees. To achieve an artificial substitution for a missing limb is necessary that the control module and the sensory module are synergistically integrated in a “real-time” framework (a time delay that is not perceivable by a potential user). To achieve this goal two equivalently important components must be integrated in a single device: real-time sensory restoration, and real-time realistic motor control of the artificial limb.

To restore a variety of sensations (e.g. touch/pressure/proprioception) in a person with limb amputation, multi- and intra-fascicular intraneural electrodes connected (through a microprocessor) to robotic limb sensors are needed. The active sites of the electrodes are used to deliver electrical stimuli to the peripheral nerves based on the readouts of artificial sensors in the limb prosthesis. By changing charge, frequency and time occurrence patterns of the electrical stimulation of singular active sites the modulation of sensation is achieved, while with multipolar stimulation (current/voltage injected in several active sites) the position/type of sensation can be changed.

The use of multi- and/or intra-fascicular intraneural electrodes represents the key feature for the homologous close-to-natural sensory restoration. This is based on the selectivity properties of neural interfaces [17, 22]. In fact, to achieve a modulation of the intensity of a sensation, a precise control on the number of fibers that encode a particular sensation in a specific region is required. This control can only be achieved through a fine modulation of the electric field distribution inside a single fascicle.

An epineural interface, as the Cuff electrode, can't achieve this fine control because the thin perineurium (the membrane that encloses the fascicles in the human nerve) is acting as an insulating structure [23] that creates a barrier effect. The result of stimulating from outside these structures is an on-off effect in which either none or many fascicles are simultaneously stimulated; an increase in the injected charge would result in the recruitment of other fascicles, thus changing the location and/or type of sensation preventing any type of strength modulation (FIG. 2). On the other side, the high intrafascicular selectivity of intraneural devices ensures a quasi-linear recruitment of fibers inside each of targeted fascicles enabling an actual modulation of the fiber population recruited. If the intrafascicular device is able to target multiple fascicles (like the TIME electrode [25] being a multi- and intra-fascicular intraneural interface) then each active site could selectively control type/extension/location/strength of the sensations. In FIG. 2 is reported a comparison of the stimulation of the nerve produced with a multi- and intra-fascicular intraneural multichannel electrode and with an epineural electrode. Presented results come from a computer simulation, executed on a realistic human nerve model. It can be noticed that, by modulating the amplitude of the stimulation, the nerve fibers recruitment occurs within a targeted fascicle in the case of the intraneural electrode and on several untargeted fascicles, before the targeted one, in the case of the epineural electrode.

A realistic motor control strategy can only be efficiently achieved if the user is able to naturally control both the movement type and its velocity/force. For this approach a smart integration between muscular and neural signals may be used. Within the hybrid framework for motor control, superficial EMG (sEMG) or intramuscular EMG (iEMG) signals are used to decode different grasps and/or movements while neural signals, Multi or Single Units, can be used to control force/velocity of a selected grasp/movement. Alternatively, the sole ENG or EMG signals can be exploited to implement the full control of the grasps/movements and their force/velocity.

The invention is based on the assumption that the user is able to exploit the dynamic sensory information induced by the intrafascicular neural stimulation, that is triggered by the transformation of readings from the sensors of the limb prosthesis, during real-time simultaneous control of a dexterous prosthesis, to adaptively modulate grasps and/or joint movements force/velocity, thus closing the user-prosthesis loop (FIG. 3).

In one embodiment of the invention the system comprises motor commands achieved by means of direct control of (surface or intramuscular) electromyographic signals and a prosthetic limb controller.

In another embodiment the system comprises motor commands achieved by means of pattern recognition of (surface or intramuscular) electromyographic signals and a prosthetic limb controller.

In another embodiment the system comprises motor commands achieved by means of pattern recognition of (surface or intramuscular) electromyographic signals for grasp/movement selection and direct control of force/velocity and a prosthetic limb controller.

In another embodiment the system comprises motor commands achieved by means of direct control of electroneurographic signals and a prosthetic limb controller.

In another embodiment the system comprises motor commands achieved by means of pattern recognition of electroneurographic signals and a prosthetic limb controller.

In another embodiment the system comprises motor commands achieved by means of pattern recognition of electroneurographic signals for grasp/movement selection and direct control of force/velocity and a prosthetic limb controller.

In another embodiment the system comprises motor commands achieved by means of direct control of hybrid combination of (surface or intramuscular) electromyographic and electroneurographic signals and a prosthetic limb controller.

In another embodiment the system comprises motor commands achieved by means of pattern recognition of (surface or intramuscular) electromyographic and electroneurographic signals and a prosthetic limb controller.

In another embodiment the system comprises motor commands achieved by means of pattern recognition of (surface or intramuscular) electromyographic signals for grasp/movement selection and direct control of force/velocity through electroneurographic signals and a prosthetic limb controller.

### BRIEF DESCRIPTION OF THE FIGURES

FIG. 1: Functional and topographical organization of peripheral nerves and multi- and intra-fascicular electrodes implant. The neural fibers are organized in the peripheral nerves in fascicles that bring specific informations. This is showed (A) in a transversal section of the median nerve extracted at the elbow [31]. To take into account this characteristic the multi- and intra-fascicular electrode is implanted transversally in the nerve (B).

FIG. 2: Stimulation by intraneural and epineural electrodes. Intraneural stimulation is more selective than the extranueral one. On the left is the result of the stimulation of a targeted fascicle by means of a multi- and intra-fascicular intraneural electrode passing into it. On the right the one when the stimulating electrode is outside the nerve (like in the case of cuffs). The latter is unable to activate the targeted fascicle without eliciting also the other untargeted, that could be conducting undesired type of sensation.

FIG. 3: General description of two embodiments according to the invention. Parts A and D of FIG. 3 show two preferred embodiments of the invention. In particular, a robotic limb prosthesis (a hand in this case) is attached to the user through a functionalized socket (A) with sEMG electrodes to acquire remnant muscular activity of the amputee. The signals are processed, decoded and the proper motor commands are sent to the robot. Force sensors embedded in the robot are transformed in stimulation signals (modulating the charge in this case) and sent to the nerve (Median and Ulnar Nerves (B and C) in this case) where transversal intrafascicular electrodes are implanted. In (D) the embodiment of the invention for the lower limb case is depicted. The intraneural stimulation of the sciatic nerve is performed, based on the sensors implemented in the artificial foot (or sock).

FIG. 4: Interaction between the components of the system according to the invention.

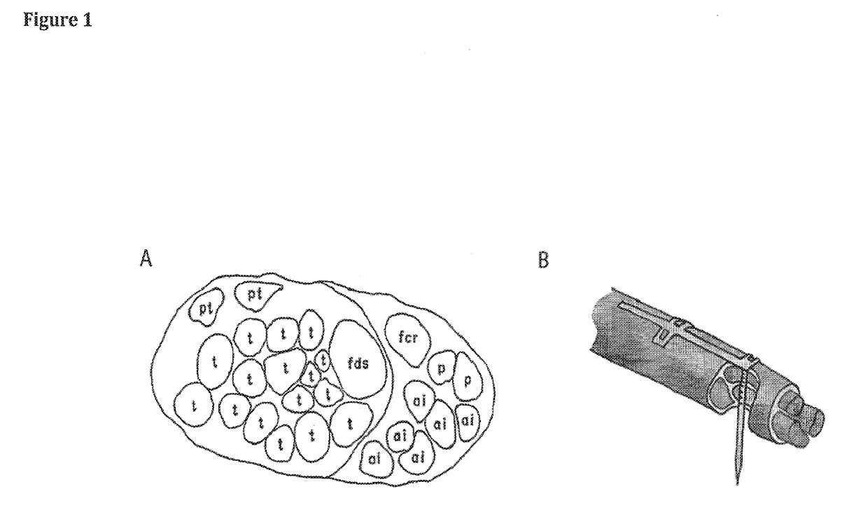

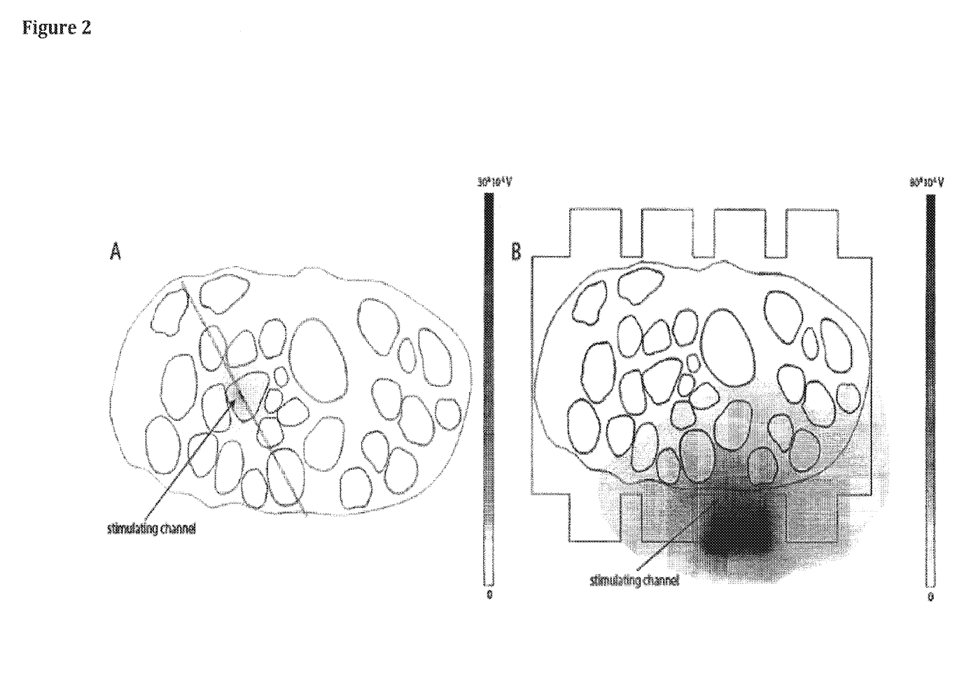

### DETAILED DESCRIPTION OF THE INVENTION

The invention will be better understood in the following text, in a detailed description and with non-limiting examples.

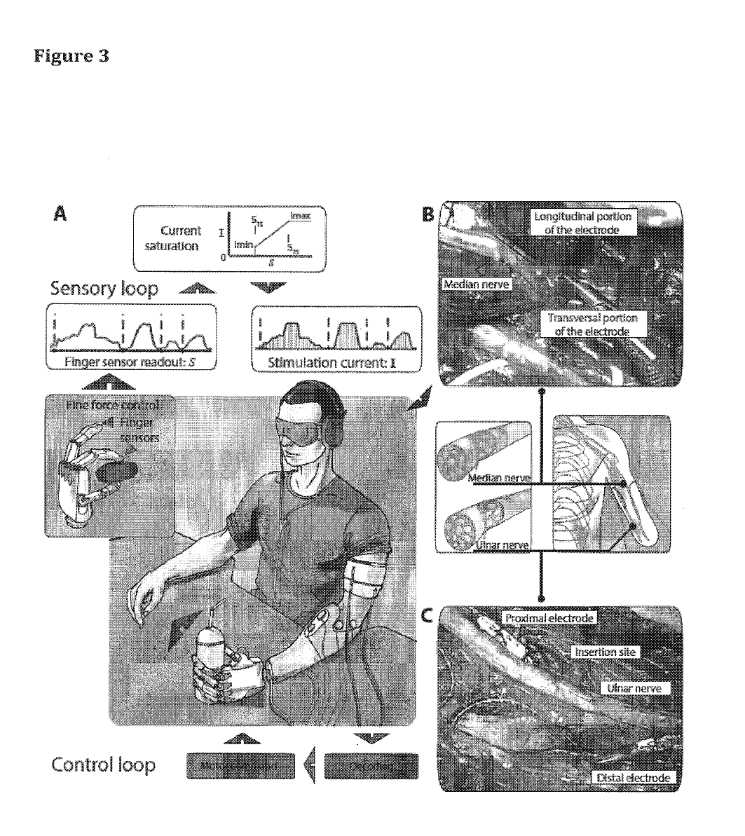

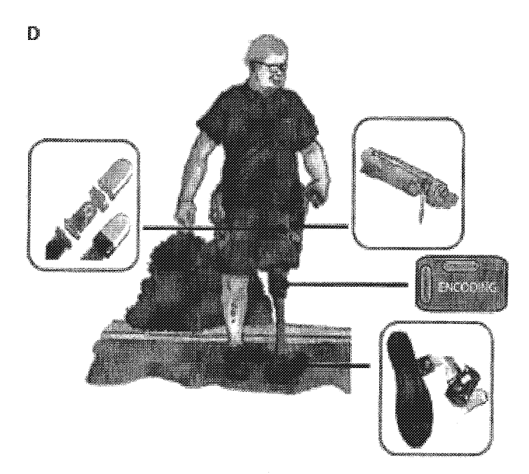

FIG. 3 illustrates the way the system controls the motion of prosthetic robotic limb and restores tactile and proprioceptive sensations in amputees using force/pressure/position/angular sensors from the finger/fingertips/palm/joints/foot of a prosthetic robotic limb.

The system is constituted by a robotic limb with embedded sensors or provided with a sensorized glove (or sock), a superficial/implantable stimulator, EMG electrodes mounted in a socket or inserted in the amputee remnant muscles, a signal conditioner and multichannel intrafascicular electrodes. The robotic limb must be connectable to the socket.

A microprocessor, cable/wireless connected to the robotic limb and to the stimulator, handles the acquisition of the EMG/ENG signals and uses them for the control of the robotic limb. Furthermore, this device reads the signals from the pressure/force/angular/position sensors and uses them to drive the stimulator for current/voltage injection to the peripheral nervous system of the amputee.

### Components of the System

1. Robotic limb

The robotic limb is comprised by several features:
i) The robotic limb should have one or preferably more degrees of freedom, and be equipped with at least one, preferably more, touch-pressure sensors and angular sensors on/within the finger/fingertips/palm/joints/foot. In the case the robotic limb is not provided with sensors, a sensorized glove (or sock) can be put over it.
ii) In the case of more proximal (e.g. above the elbow or the knee) amputations, it should be equipped with controllable wrist/elbow/knee/ankle, provided with sensors.
iii) The functionalized socket to be adapted to the stump should include monopolar or bipolar surface EMG electrodes or multipolar electrode arrays for electromyographic activity.
iv) In the case of the intramuscular EMG step iii) is not required.
v) In the case of targeted-muscle re-innervation (TMR) users, the electrode arrays can be placed over the targeted breast/leg muscles.
2. Artificial sensors within robotic limb or glove (or sock)

Any prosthetic hand/foot/arm/leg with force/pressure/angular/position sensors in/on the fingers/finger tips/palm/wrist/elbow/knee/ankle can be used for the method. The sensors must give a continuous measurement with a sampling frequency of minimum 10 Hz, in Pa (for pressure sensors) or N (for force-tension sensors). Position and/or angular sensors of the fingers/joints are to be used for providing proprioceptive sensations. The sensors should have capacity to detect the area of contact and precise timing of its dynamic change.
3. Implanted Electrodes

Multi- and intra-fascicular electrodes (provided with bio-compatible cables and connectors) to be implanted in the Median and/or Ulnar and /or Radial nerves, within the residual arm, or in the case of TMR amputees within the transferred nerves. For the lower limb to be implanted within femoral/sciatic/tibial residual nerves, or in the case of lower limb TMR amputees within the transferred nerves.
4. Current/voltage Stimulator

The stimulator can be transcutaneously connected to the electrodes or can be implantable. It must have at least 2 independent (in terms of the all stimulation parameters: amplitude, pulsewidth, frequency) channels, being preferable the solution with many channels.
5. Signal Conditioner

A signal conditioner picks-up the signals coming from the ENG, EMG electrodes, then amplifies and filters them. This device has to be connected wireless or wired with the ENG, EMG electrodes. Then, it has to send the amplified signals to the processor. The signal amplifier can be implantable or external to the body.
6. Microprocessor

A microprocessor unit will manage:
i) a) The acquisition of biological signals (muscular and neural) b) processing and c) decoding the voluntary intention of user, in order to control the motion of the robotic limb.
ii) a) The acquisition of robotic limb sensors readouts, b) Processing and c) Encoding of the information, in order to control the stimulator for the sensory restoration.

### Methods

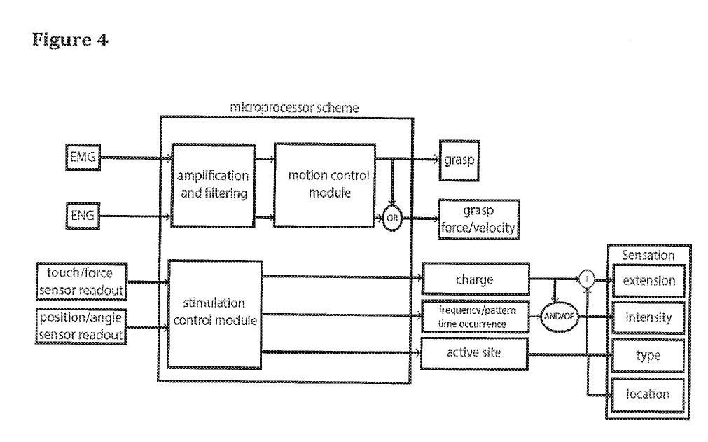

The system schematically illustrated in FIG. 4 is described below.
1. Hybrid Control of the artificial limb

The hybrid control strategy of the artificial limb is composed of two steps that could work as independent or synergistic modules:
i) Electromyographic (EMG) control module: the control is implemented through a classifier, which recognizes the EMG activity at fixed steps (for example 100 ms) and decode the type of grasp/movement that the user wants to perform. The amputee can in every moment change the muscular activity and switch from one to another type of grasp/movement. This control should work with different electrodes for EMG recordings that could be surface electrodes, surface electrodes arrays or intramuscular electrodes.
ii) Electroneurographic (ENG) control module: the ENG signal is used in combination with the EMG module to control the grasping force/velocity of the selected grasp by working in synergy with it.
iii) In the case of the absence or impossibility of recording the ENG signal, the velocity of the motors will be controlled by the EMG control module.
2. Transfer function to the Nervous System.

The readout of the sensors embedded in the prosthetic limb or the glove (or sock) is used as an input for the delivery of afferent neural stimulation. The system can select and modulate 4 (or more) different characteristics of sensations:
i) type of sensation
ii) strength (intensity)
iii) location over phantom limb
iv) spatial extension

### Type of Sensation

The type of sensation is selected by the stimulation of particular active sites of the peripheral nerve interface. In the case of multi- and intra-fascicular electrodes each usable active site will in fact elicit a specific type of sensation. These sensations could be touch/pressure and proprioception among others.

### Strength of Sensation

The strength of sensation can be modulated through the use of charge (amplitude/pulse width), frequency and pattern of stimulus time occurrence modulation. In the case of the charge modulation an intrafascicular device ensure a quasi-linear dynamic relationship strength-amplitude.

The relationship between the tension-touch hand sensors readout and the charge of the stimulation current pulses could be implemented (nut not limited to) as follows:

c=(cₘₐₓ−cₘᵢₙ)*(s−s₁₅)/(s₇₅−s₁₅)+c_{min, when} s₁₅≤s≤s₇₅;

c=0, when s_{15<s;}

c=cₘₐₓ, when s>s₇₅;

where:

c is the amplitude of stimulation current,

s is the sensor readout,

s₁₅ and s₇₅ represent 15% and 75% respectively of the maximum range of the sensor readout, which characterize, respectively, the contact point of the robotic hand with an object and a value tuned to exploit the full range of sensations for all objects, cₘᵢₙ and cₘₐₓ are the stimulation current amplitudes that elicited, respectively, the minimum and the maximum (i.e., below pain threshold) touch sensations, as reported by the subject. The frequency of the stimulation in this example is fixed.

An analogous relation can be implemented in the case of frequency modulation:

f=(fmax−fmin)*(s−s15)/(s75−s15)+fmin, when s15≤s≤s75;

f=0, when s15<s;

f=cmax, when s>s75;

In this case f is the frequency of the stimulation. The current amplitude is fixed and set to a value that elicits a sensation in the middle between minimum and below pain threshold perceived sensations.

In the case of the modulation of the time occurrence (TO) of the stimulation pattern, several relations (sensors readouts-TO) can be implemented. The TO is defined as the time delay between a pattern of stimulation and the successive one.

A linear (sensors readouts-TO) relation is defined as follows:

TO=−(TOmax−TOmin)*(s−s15)/(s75−s15)+TOmax, s15≤s≤s75;

No stimulation, s15<s;

TO=TOmax, s>s75;

In this case, the current amplitude is fixed and set to a value that elicits a sensation in the middle between minimum and below pain threshold perceived sensation.

In all the presented cases, other possible relations can be implemented between the sensors readouts and the stimulation (e.g. sigmoid or Poisson relations). Moreover, similar relations can be implemented in the case a voltage stimulator and other types of sensors are used in the bidirectional prosthesis.

Charge, frequency and pattern time occurrence modulation can be implemented and exploited together or separately in the bidirectional prosthesis.

### Location Over Phantom Limb

This characteristic is controlled by the spatial location of the electrodes: different active sites of the electrode, debt to transversal somato-topography of peripheral nerves, will elicit the sensations over different areas of the missing limb. Intra-fascicular electrodes ensure a localized sensation per active site, being able to stimulate single nerve fascicles, thus each active site could control a specific and delimited sensory area. For example in the case of hand/arm amputation an electrode implanted in the residual median nerve will elicit the sensations over first three fingers and underlying palm area. Electrode implanted in residual ulnar nerve will elicit the sensations over last two fingers and underlying palm area. Finally, electrode implanted in radial nerve will elicit sensations over wrist and dorsal hand. In the lower limb the sciatic nerve stimulation will enable for the coverage of the main part of the phantom foot sensations.

### Spatial Extension

This characteristic is controlled by a combination of the spatial location and amplitude modulation of the active sites. Several active sites of electrode, that elicit different sensations, will be used together in multipolar stimulation strategies to move the sensation over different hand areas (e.g. the sensations elicited in thumb and index finger could be combined so to obtain the feeling of the palm that is under these two fingers).
3. Real-time integration microprocessor unit

The recording of biological signals, features extraction, and final decoding of the user intended grasp/movement will be done in parallel with sensors readout, and transformation for encoding of the sensation. All the computation should be performed in timing within 100 msec that is essential to be imperceptible to the user.

Of course the invention is not limited to the examples presented previously.

Any suitable highly selective neural fiber stimulation tool can be used, for instance the use of optogenetic technologies [26] or a combination of electrical and optical stimulation [27].

The number of EMG and/or sensory feedback electrodes is also not limited, the main objective being to provide a highly selective stimulation between two adjacent fascicules or between the axons located within the same fascicule.

### REFERENCES

1. K. T. Reilly, C. Mercier, M. H. Schieber, A. Sirigu, Persistent hand motor commands in the amputees' brain. Brain 129, 2211-23 (2006).
2. A. W. Goodwin, H. E. Wheat, Sensory signals in neural populations underlying tactile perception and manipulation. Annu Rev Neurosci. 27, 53-77 (2004).
3. W. Schady, J. L. Ochoa, H. E. Torebjork, L. S. Chen, Peripheral projections of fascicles in the human median nerve. Brain 106, 745-760 (1983).
4. P. Marchettini, M. Cline, J. L. O choa, Innervation territories for touch and pain afferents of single fascicles of the human ulnar nerve. Mapping through intraneural microrecording and microstimulation. Brain 113, 1491-500 (1990).
5. W. Schady, S. Braune, S. Watson, H. E. Torebjörk, R. Schmidt, Responsiveness of the somatosensory system after nerve injury and amputation in the human hand. Ann. Neurol. 36, 68-75 (1994).
6. D. Atkins, D. Heard, W. Donovan, Epidemiologic overview of individuals with upper-limb loss and their reported research priorities. J Prosthet. Orthot. 8, 2-11 (1996).
7. E. Biddiss, D. Beaton, T. Chau, Consumer design priorities for upper limb prosthetics. Disabil. Rehabil. Assist. Technol. 2, 346-57 (2006).
8. M. C. Carrozza, G. Cappiello, S. Micera, B. B. Edin, L. Beccai L., et al., Design of a cybernetic hand for perception and action. Biol. Cybern. 95, 629-644 (2006).
9. M. S. Johannes, J. D. Bigelow, J. M Burck, S. D. Harshbarger, M. V. Kozlowski, T. Van Doren, An Overview of the Developmental Process for the Modular Prosthetic Limb. Johns Hopkins APL Tech Dig, 30, 207-216 (2011).
10. C. Connolly, Prosthetic hands from Touch Bionics. Industrial Robot. 35, 290-293 (2008).
11. T. A. Kuiken, L. A. Miller, R. D. Lipschutz, B. A. Lock, K. Stubblefield, et al., Targeted reinnervation for enhanced prosthetic arm function in a woman with a proximal amputation: a case study. Lancet 369, 371-380 (2007).
12. T. A. Kuiken, G. Li, B. A. Lock, R. D. Lipschutz, L. A. Miller, et al., Targeted muscle reinnervation for real-time myoelectric control of multifunction artificial arms. JAMA 301, 619-628 (2009).
13. P. D. Marasco, A. E. Schultz, T. A. Kuiken, Sensory capacity of reinnervated skin after redirection of amputated upper limb nerves to the chest. Brain 132, 1441-8 (2009).
14. T. A. Kuiken, P. D. Marasco, B. Lock, R. Harden, J. Dewald, Redirection of cutaneous sensation from the hand to the chest skin of human amputees with targeted reinnervation. Proc. Natl. Acad. Sci. USA 104, 20061-6 (2007).
15. R. Kristeva-Feige, S. Rossi, V. Pizzella, L. Lopez, S. N. Erne, J. Edrich, P. M. Rossini A neuromagnetic study of movement-related somatosensory gating in the human brain. Exp Brain Res. 107, 504-14 (1996).
16. L Hargrove, A, Simon; A. Young; R Lipschutz, S, Finucane, D. Smith, T. Kuiken, Robotic leg control with EMG decoding in an amputee with nerve transfers. New England Journal of Medicine. 369, 1237-1242 (2013)
17. X. Navarro, T. Krueger, N. Lago, S. Micera, T. Stieglitz, et al., A critical review of interfaces with the peripheral nervous system for the control of neuroprostheses and hybrid bionic systems. J. Peripher. Nerv. System 10, 229-258 (2005).
18. G. S. Dhillon, T. B. Kruger, J. S. Sandhu, K. W. Horch, Effects of short-term training on sensory and motor function in severed nerves of long-term human amputees. J Neurophysiol. 93, 2625-2633 (2005)
19. G. S. Dhillon, S. M. Lawrence, D. T. Hutchinson, K. W. Horch, Residual function in peripheral nerve stumps of amputees: implications for neural control of artificial limbs J Hand Surg Am. ;29, 605-15 (2004)
20. P. M. Rossini, S. Micera, A. Benvenuto, J. Carpaneto, G. Cavallo, et al,. Double nerve intraneural interface implant on a human amputee for robotic hand control. Clin. Neurophysiol. 121, 777-783 (2010).
21. X. Jia, M. A. Koenig, X. Zhang, J. Zhang, T. Chen, et al., Residual motor signal in longterm human severed peripheral nerves and feasibility of neural signal controlled artificial limb. J. Hand Surg. Am. 32, 657-66 (2007).
22. J. Badia, T. Boretius, D. Andreu, C. Azevedo-Coste, T. Stieglitz, and X. Navarro, “Comparative analysis of transverse intrafascicular multi-channel, longitudinal intrafascicular and multipolar cuff electrodes for the selective stimulation of nerve fascicles,” J. Neural Eng., vol. 8, no. 3, (2011).
23. S. Raspopovic, M. Capogrosso, and S. Micera, “A computational model for the stimulation of the rat sciatic nerve using a transverse intrafascicular multichannel electrode,” IEEE Trans. Neural Syst. Rehabil. Eng., vol. 19, no. 4, pp. 333-344, Aug. (2011).
24. T. R. Farrell, R. F. Weir, The optimal controller delay for myoelectric prostheses. IEEE Trans. Neural. Syst. Rehabil. Eng. 15, 111-8 (2007).
25. T. Boretius , J. Badia , A. Pascual-Font, M. Schuettler, X. Navarro, K Yoshida, T. Stieglitz, A transverse intrafascicular multichannel electrode (TIME) to interface with the peripheral nerve. Biosensors and Bioelectronics, 26(1), 62-69, 2010.
26. M. E. Llewellyn, K. R. Thompson, K. Deisseroth, S. L. Delp, Orderly recruitment of motor units under optical control in vivo Nature Medicine 16, 1161-1165, 2010.
27. Duke AR¹, Peterson E, Mackanos M A, Atkinson J, Tyler D, Jansen E D. Hybrid electro-optical stimulation of the rat sciatic nerve induces force generation in the plantarflexor muscles, J Neural Eng, 9(6):066006. 2012.
28. Cipriani, Christian, Marco D'Alonzo, and Maria Chiara Carrozza. “A miniature vibrotactile sensory substitution device for multifingered hand prosthetics.” Biomedical Engineering, IEEE Transactions on 59.2 (2012): 400-408.
29. A. Y. j Szeto e F. A. Saunders, «Electrocutaneous Stimulation for Sensory

Communication in Rehabilitation Engineering», IEEE Trans. Biomed. Eng., vol. BME-29, n. 4, pagg. 300-308, apr. 1982.
30. Kuhn, Andreas, et al. “The influence of electrode size on selectivity and comfort in transcutaneous electrical stimulation of the forearm.” Neural Systems and Rehabilitation Engineering, IEEE Transactions on 18.3 (2010): 255-262.
31. Jabaley, Michael E., William H. Wallace, and Frederick R. Heckler. “Internal topography of major nerves of the forearm and hand: a current view.” The Journal of hand surgery 5.1, (1980).

1-12. (canceled)

13. A method performed on a closed-loop real-time limb neuroprosthetic system, the system including an artificial limb or a sensorized glove or sock, a microprocessor, sensors, a signal conditioner, a stimulator, an electromyography (EMG) electrode, and a sensory feedback electrode, the method comprising the steps of:
implanting the sensory feedback electrode transversally in an intact and healthy portion of a nerve; and
modulating an intensity of a sensory feedback from the sensory feedback electrode by changing an injected charge with respect to a readout of the sensors, the sensors being embedded in the artificial limb or in the sensorized glove or sock.

14. The method according to claim 13, wherein the step of modulating further comprises:
modulating the intensity of the sensory feedback by changing a stimulation frequency with respect to the readout of the sensors that are embedded in the artificial limb or in the sensorized glove or sock.

15. The method according to claim 13, wherein the step of modulating further comprises:
modulating the intensity of the sensory feedback by changing a time occurrence of a stimulation pattern with respect to the readout of the sensors that are embedded in the artificial limb or in the sensorized glove or sock.

16. The method according to claim 13, wherein the step of modulating further comprises:
modulating the intensity of the sensory feedback by a multi-polar stimulation with respect to the readout of the sensors embedded in the artificial limb or in the sensorized glove or sock.

17. The method according to claim 13, wherein the step of modulating further comprises:
modulating at least one of a type and location of the sensory feedback by changing the stimulation of an active site of a peripheral nerve interface with respect to the readout of the sensors embedded in the artificial limb or in the sensorized glove or sock.

18. The method according to claim 13, wherein the step of modulating further comprises:
modulating at least one of a type and location of the sensory feedback by multi polar stimulation with respect to the readout of the sensors embedded in the artificial limb or in a sensorized glove or sock.

19. The method according to claim 13, wherein implanting the sensory feedback electrode is performed in a way as to differentiate a fiber recruitment of a fascicle.

20. The method according to claim 13, wherein implanting the sensory feedback electrode is performed in a way as to differentiate a fiber recruitment of two fascicles.

21. The method according to claim 13, wherein implanting the sensory feedback electrode is performed through insertion within a nerve fascicle.

22. The method according to claim 13, wherein the step of implanting the sensory feedback electrode includes an implanting of the sensory feedback electrode in a transversal section of a median nerve extracted at the elbow.
